(19) United States
(12) Patent Application Publication
(10) Pub. No.: US 2025/0391868 A1
(43) Pub. Date: Dec. 25, 2025

(54) ADDITIVE FOR POSITIVE ELECTRODE, POSITIVE ELECTRODE INCLUDING THE SAME, AND RECHARGEABLE LITHIUM BATTERY INCLUDING THE SAME

(71) Applicant: SAMSUNG SDI CO., LTD., Yongin-si (KR)

(72) Inventors: Hyunwoo KIM, Yongin-si (KR); Seung-Hun HAN, Yongin-si (KR); Moonju CHO, Yongin-si (KR); Seonghyun LIM, Yongin-si (KR); SooChan KIM, Yongin-si (KR); Myunghoon KIM, Yongin-si (KR)

(21) Appl. No.: 19/311,856

(22) Filed: Aug. 27, 2025

Related U.S. Application Data
(63) Continuation of application No. 19/204,924, filed on May 12, 2025, now abandoned.

(30) Foreign Application Priority Data
Jun. 3, 2024 (KR) 10-2024-0072376

(51) Int. Cl. H01M 4/62; C07D 249/08; H01M 4/02; H01M 4/04; H01M 4/40

(52) U.S. Cl. CPC H01M 4/621; C07D 249/08; H01M 4/0407; H01M 4/405; H01M 2004/027; H01M 2004/028

(57) ABSTRACT

An additive for a positive electrode for a rechargeable lithium battery is disclosed. A positive electrode active material slurry may include a functional additive, a positive electrode active material, a conductive material, and a binder. The functional additive may include a compound containing a triazole group.

### CROSS-REFERENCE TO RELATED APPLICATION

This application is a continuation of U.S. patent application Ser. No. 19/204,924, filed on May 12, 2025, which claims priority to and the benefit of Korean Patent Application No. 10-2024-0072376, filed on Jun. 3, 2024, in the Korean Intellectual Property Office, the entire disclosure of each of which is incorporated herein by reference.

### BACKGROUND

### 1. Field

One or more embodiments of the present disclosure relate to an additive for a positive electrode for a rechargeable lithium battery.

### 2. Description of the Related Art

With the rapid spread of electronic devices that use batteries (e.g., battery-powered electronic devices), such as mobile phones, laptop computers, and/or electric vehicles, the demand for rechargeable batteries with relatively high energy density and high capacity is rapidly increasing (growing). Accordingly, research and development to improve (enhancing) the performance of rechargeable batteries, e.g., rechargeable lithium batteries, is being actively conducted.

A rechargeable lithium battery is a battery that includes a positive electrode and a negative electrode, containing active materials capable of intercalation and deintercalation of lithium ions, and an electrolyte, and that produces electrical energy through the oxidation and reduction reactions if (e.g., when) lithium ions are intercalated into and deintercalated from the positive electrode and/or the negative electrode.

### SUMMARY

One or more aspects of embodiments of the present disclosure are directed toward a positive electrode that includes a functional additive.

One or more aspects of embodiments of the present disclosure are also directed toward a method for fabricating a rechargeable lithium battery by adding a functional additive.

Additional aspects will be set forth in part in the description which follows and, in part, will be apparent from the description, or may be learned by practice of the presented embodiments of the disclosure.

According to one or more embodiments of the present disclosure, a positive electrode active material slurry may include a functional additive, a positive electrode active material, a conductive material, and a binder, and the functional additive may include a compound containing a triazole group.

According to one or more embodiments of the present disclosure, a rechargeable lithium battery may include a positive electrode, a negative electrode, and an electrolyte layer between the positive electrode and the negative electrode, where the positive electrode may include a positive electrode current collector and a positive electrode active material layer that is on the positive electrode current collector, the negative electrode may include a negative electrode current collector and a negative electrode active material layer that is on the negative electrode current collector, and the positive electrode active material layer may include the positive electrode active material slurry.

According to one or more embodiments of the present disclosure, a method for fabricating a rechargeable lithium battery may include manufacturing a positive electrode, manufacturing a negative electrode, and combining the positive electrode and the negative electrode to fabricate a battery, where the manufacturing of the positive electrode may include mixing a positive electrode active material, a conductive material, a binder, and a functional additive to prepare a positive electrode active material slurry, and applying the positive electrode active material slurry on a current collector to form or provide a positive electrode active material layer, and the functional additive may include a compound containing a triazole group.

### BRIEF DESCRIPTION OF THE DRAWINGS

The accompanying drawings are included to provide a further understanding of disclosure, and are incorporated in and constitute a part of this specification. The drawings illustrate embodiments of the present disclosure and, together with the description, serve to illustrate principles of disclosure.

FIG. 1 is a simplified conceptual diagram illustrating a rechargeable lithium battery according to one or more embodiments of the present disclosure.

FIGS. 2-5 are simplified cross-sectional views illustrating rechargeable lithium batteries according to one or more embodiments of the present disclosure.

FIG. 6 is a graph illustrating viscosity evaluation results of positive electrode active material slurries according to the Examples and Comparative Examples in the present disclosure.

FIG. 7 is scanning electron microscope (SEM) images of the surface of a positive electrode active material according to the Comparative Example in the present disclosure.

FIG. 8 is scanning electron microscope (SEM) images of the surface of a positive electrode active material according to the Example in the present disclosure.

FIG. 9 is a graph illustrating resistance evaluation results of positive electrode active material slurries according to the Examples and Comparative Example in the present disclosure.

FIG. 10 is transmission electron microscope (SEM) images of a positive electrode active material according to the Comparative Example in the present disclosure.

FIG. 11 is transmission electron microscope (SEM) images of a positive electrode active material according to the Example in the present disclosure.

FIGS. 12A-12C are XPS analysis results of positive electrodes according to the Example and Comparative Example in the present disclosure.

### DETAILED DESCRIPTION

In order to sufficiently understand the configurations and aspects of the present disclosure, one or more embodiments of the present disclosure will be described with reference to the accompanying drawings. It should be noted, however, that the present disclosure is not limited to the following example embodiments and may be implemented in one or more suitable forms. Rather, the example embodiments are provided only to illustrate the subject matter of the present disclosure and let those having ordinary skill in the art fully understand the scope of the present disclosure.

As utilized herein, the terms, “and/or” and “or,” may include any and all combinations of one or more of the associated listed items. Expressions, such as “at least one of,” when preceding a list of elements, modify the entire list of elements and do not modify the individual elements of the list.

It will be further understood that the terms, “comprise,” “include,” or “have/has,” when utilized in the present disclosure, specify the presence of stated features, integers, steps, operations, elements, and/or components, but do not preclude the presence or addition of one or more other features, integers, steps, operations, elements, components, and/or groups thereof. The “/” utilized below may be interpreted as “and” or as “or” depending on the situation.

In the context of the present disclosure and unless otherwise defined, the terms, “use,” “using,” and “used,” may be considered synonymous with the terms “utilize,” “utilizing,” and “utilized,” respectively.

As utilized herein, the term, “about,” or similar terms are used as terms of approximation and not as terms of degree, and are intended to account for the inherent deviations in measured or calculated values that would be recognized by those of ordinary skill in the art. “About” or “approximately,” as used herein, is also inclusive of the stated value and means within an acceptable range of deviation for the particular value as determined by one of ordinary skill in the art, considering the measurement in question and the error associated with measurement of the particular quantity (e.g., the limitations of the measurement system). For example, “about” may mean within one or more standard deviations, or within ±30%, 20%, 10%, or ±5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

In the present disclosure, it will be understood that, if (e.g., when) an element is referred to as being on another element, the element may be directly on the other element or intervening elements may be present therebetween. In contrast, if (e.g., when) an element is referred to as being “directly on” another element, there are no intervening elements present. In the drawings, thicknesses of some components may be exaggerated for effectively illustrating the technical contents. Like reference numerals refer to like elements throughout the present disclosure, and duplicative descriptions thereof may not be provided for conciseness.

Unless otherwise specially noted in the present disclosure, the singular forms “a,” “an,” and “the” are intended to include the plural forms as well, unless the context clearly indicates otherwise. Further, the utilization of “may” when describing embodiments of the present disclosure refers to “one or more embodiments of the present disclosure.” In addition, unless otherwise specially noted, the phrase “A or B,” “A and/or B,” or “A/B” may indicate “A but not B,” “B but not A,” and “A and B.” The terms, “comprises/includes” and/or “comprising/including,” used in the present disclosure do not exclude the presence or addition of one or more other components.

As used herein, the term, “combination thereof,” may refer to a mixture, a stack, a composite, a copolymer, an alloy, a blend, or a reaction product of the constituents.

Unless otherwise specially defined in the present disclosure, a particle diameter may be an average particle diameter. Also, a particle diameter refers to an average particle diameter (D50). D50 refers to the average diameter (or size) of particles whose cumulative volume corresponds to 50 vol % in the particle size distribution (e.g., cumulative distribution), and refers to the value of the particle size corresponding to 50% from the smallest particle if (e.g., when) the total number of particles is 100% in the distribution curve accumulated in the order of the smallest particle size to the largest particle size. In the present disclosure, if (e.g., when) particles are spherical, “diameter” indicates an average particle diameter, and if (e.g., when) the particles are non-spherical, the “diameter” indicates a major axis length. The average particle diameter (D50) may be measured by a method widely suitable to those skilled in the art, for example, by a particle size analyzer, for example, HORIBA or LA-950 laser particle size analyzer, or by using a transmission electron microscope (TEM) or a scanning electron microscope (SEM). In one or more embodiments, the average particle diameter may be measured by a measurement device using dynamic light scattering, where data analysis is conducted to count the number of particles for each particle size range, and an average particle diameter (D50) value may then be obtained through calculation. Also, a laser scattering method may be utilized to measure the average particle diameter. In the laser scattering method, target particles are dispersed in a dispersion medium, then, introduced into a commercial laser diffraction particle-diameter measurement instrument (e.g., MT3000 of Microtrac), and irradiated to ultrasonic waves of about 28 kHz at an output of about 60 W, and the average particle diameter (D50) based on about 50% of particle diameter distribution may be calculated in the measurement instrument.

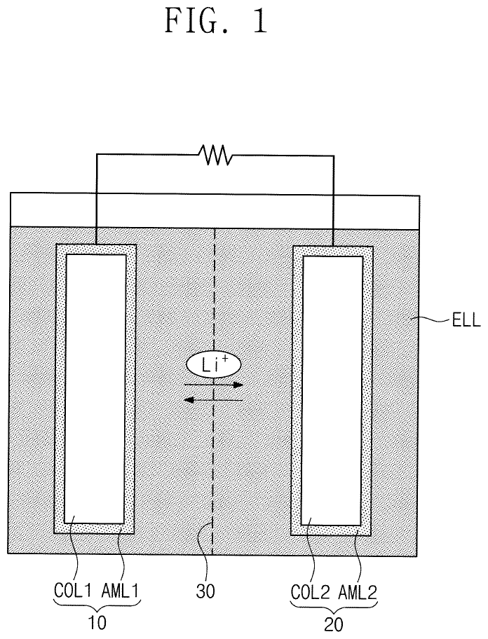

FIG. 1 is a simplified conceptual diagram of a rechargeable lithium battery according to one or more embodiments of the present disclosure. Referring to FIG. 1, a rechargeable lithium battery may include a positive electrode 10, a negative electrode 20, a separator 30, and an electrolyte solution ELL.

The positive electrode 10 and the negative electrode 20 may be spaced and/or apart (e.g., spaced apart or separated) from each other by the separator 30. The separator 30 may be arranged between the positive electrode 10 and the negative electrode 20. The positive electrode 10, the negative electrode 20, and the separator 30 may be in contact with the electrolyte solution ELL. In one or more embodiments, the positive electrode 10, the negative electrode 20, and the separator 30 may be immersed in the electrolyte solution ELL.

The electrolyte solution ELL may be a medium in which lithium ions are migrated and transferred between the positive electrode 10 and the negative electrode 20. In the electrolyte solution ELL, the lithium ions may move through the separator 30 toward one of (e.g., selected from among) the positive electrode 10 or the negative electrode 20.

A rechargeable battery including a gel polymer electrolyte (or semi-solid electrolyte) and a solid electrolyte may include an electrolyte layer. In one or more embodiments, the electrolyte layer may replace the role of the separator 30 and the electrolyte solution (ELL).

### Positive Electrode 10

The positive electrode 10 for a rechargeable lithium battery may include a current collector COL1 and a positive electrode active material layer AML1 that is on the current collector COL1. The positive electrode active material layer AML1 may include a positive electrode active material (e.g., in a form of particles) and may further include a binder and/or a conductive material (e.g., an electrically conductive material or electron conductor). The content (e.g., amount) of the positive electrode active material in the positive electrode active material layer AML1 may be in a range of about 90 wt % to about 99.5 wt % on the basis of about 100 wt % (e.g., based on 100 wt %) of the positive electrode active material layer AML1. The contents (e.g., amounts) of the binder and the conductive material may each or together be about 0.5 wt % to about 5 wt % on the basis of about 100 wt % (e.g., based on 100 wt %) of the positive electrode active material layer AML1. The positive electrode active material layer AML1 may further include a functional additive (add) as described in one or more embodiments of the present disclosure. Detailed description on the positive electrode according to one or more embodiments of the present disclosure will be illustrated. Aluminum (Al) may be used as the current collector COL1, but embodiments of the present disclosure are not limited thereto.

### Positive Electrode Active Material

The positive electrode active material in the positive electrode active material layer AML1 may include a compound (e.g., lithiated intercalation compound) that may reversibly intercalate and de-intercalate lithium. For example, the positive electrode active material may use at least one type or kind of the composite oxide of lithium and a metal that is selected from among cobalt, manganese, nickel, and/or a (e.g., any suitable) combination thereof. That is, the positive electrode active material may utilize one or more types of lithium-metal composite oxides, with the metal being chosen from cobalt, manganese, nickel, or any suitable combination of these elements.

The composite oxide may include lithium transition metal composite oxide(s), for example lithium nickel-based oxide(s), lithium cobalt-based oxide(s), lithium manganese-based oxide(s), lithium iron phosphate-based compounds, cobalt-free nickel manganese-based oxides, and/or (e.g., any suitable) combination(s) thereof.

For example, the positive electrode active material may include a compound represented by any one selected from among chemical formulae: LiₐA_{1-b}X_{b}O_{2-c}D_{c} (0.90≤a≤1.8, 0≤b≤0.5, 0≤c≤0.05), LiₐMn_{2-b}X_{b}O_{4-c}D_{c} (0.90≤a≤1.8, 0≤b≤0.5, 0≤c≤0.05), LiₐNi_{1-b-c}Co_{b}X_{c}O_{2-α}D_{α} (0.90≤a≤1.8, 0≤b≤0.5, 0≤c≤0.5, 0<α<2), LiₐNi_{1-b-c}Mn_{b}X_{c}O_{2-α}D_{α} (0.90≤a≤1.8, 0≤b≤0.5, 0≤c≤0.5, 0<α<2), LiₐNi_{b}Co_{c}L¹_{d}GeO₂ (0.90≤a≤1.8, 0≤b≤0.9, 0≤c≤0.5, 0≤d≤0.5, 0≤e≤0.1), LiₐNiG_{b}O₂ (0.90≤a≤1.8, 0.001≤b≤0.1), LiₐCoG_{b}O₂ (0.90≤a≤1.8, 0.001≤b≤0.1), LiₐMn_{1-b}G_{b}O₂ (0.90≤a≤1.8, 0.001≤b≤0.1), LiₐMn₂G_{b}O₄ (0.90≤a≤1.8, 0.001≤b≤0.1), LiₐMn_{1-g}G_{g}PO₄ (0.90≤a≤1.8, 0≤g≤0.5), Li_{(3-f)}Fe₂(PO₄)₃ (0≤f≤2), and LiₐFePO₄ (0.90≤a≤1.8).

In the foregoing chemical formulae, A may be nickel (Ni), cobalt (Co), manganese (Mn), and/or a (e.g., any suitable) combination thereof, X may be Al, Ni, Co, Mn, chromium (Cr), iron (Fe), magnesium (Mg), strontium (Sr), vanadium (V), a rare earth element, and/or a (e.g., any suitable) combination thereof, D may be oxygen (O), fluorine (F), sulfur (S), phosphorus (P), and/or a (e.g., any suitable) combination thereof, G may be Al, Cr, Mn, Fe, Mg, lanthanum (La), cerium (Ce), Sr, V, and/or a (e.g., any suitable) combination thereof, and L¹ may be Mn, Al, and/or a (e.g., any suitable) combination thereof.

For example, the positive electrode active material may be a high nickel-based positive electrode active material having the nickel content (e.g., amount) of about 80 mol % or more, about 85 mol % or more, about 90 mol % or more, about 91 mol % or more, or about 94 mol % or more, and about 99 mol % or less on the basis of about 100 mol % (e.g., based on 100 mol %) of metals excluding lithium in a lithium transition metal composite oxide. The high nickel-based positive electrode active material may achieve high capacity and thus may be applied to a high-capacity and high-density rechargeable lithium battery.

### Positive Electrode Binder

The binder may serve to improve attachment of positive electrode active material particles to each other and also to improve attachment of the positive electrode active material to the current collector COL1. The binder may include, for example, one or more selected from among polyvinyl alcohol, carboxymethyl cellulose, hydroxypropyl cellulose, diacetyl cellulose, polyvinyl chloride, carboxylated polyvinyl chloride, polyvinyl fluoride, an ethylene oxide-containing polymer, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, and nylon, but embodiments of the present disclosure are not limited thereto.

### Positive Electrode Conductive Material

The conductive material (e.g., an electrically conductive material or electron conductor) may be used to provide an electrode with conductivity (e.g., electrical conductivity), and any suitable conductive materials without causing chemical change (e.g., that does not cause an undesirable chemical change) of a battery may be used as the conductive material to constitute the battery. The conductive material may include, for example, a carbon-based material, such as natural graphite, artificial graphite, carbon black, acetylene black, Ketjen black, carbon fiber, carbon nano-fiber, and/or carbon nano-tube, a metal-based material with a metal powder or metal fiber type or kind, containing copper, nickel, aluminum, silver and/or the like, a conductive polymer (e.g., an electrically conductive polymer), such as a polyphenylene derivative, and/or a (e.g., any suitable) mixture thereof.

### Negative Electrode 20

The negative electrode 20 for a rechargeable lithium battery may include a current collector COL2 and a negative electrode active material layer AML2 that is on the current collector COL2. The negative electrode active material layer AML2 may include a negative electrode active material (e.g., in a form of particles) and may further include a binder and/or a conductive material (e.g., an electrically conductive material or electron conductor).

For example, the negative electrode active material layer AML2 may include about 90 wt % to about 99 wt % of the negative electrode active material, about 0.5 wt % to about 5 wt % of the binder, and about 0 wt % to about 5 wt % (e.g., at most 5 wt %) of the conductive material on the basis of about 100 wt % (e.g., based on 100 wt %) of the negative electrode active material layer AML2.

### Negative Electrode Binder

The binder may serve to improve attachment of the negative electrode active material particles to each other (e.g., to attach the negative electrode active material particles well to each other) and also to improve attachment of the negative electrode active material to the current collector COL2 (e.g., to attach the negative electrode active material well to the current collector COL2). The binder may include a non-aqueous (e.g., water-insoluble) binder, an aqueous (e.g., water-soluble) binder, a dry binder, and/or a (e.g., any suitable) combination thereof.

The non-aqueous binder may include polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, polyamide-imide, polyimide, and/or a (e.g., any suitable) combination thereof.

The aqueous binder may be selected from among a styrene-butadiene rubber, a (meth)acrylated styrene-butadiene rubber, a (meth)acrylonitrile-butadiene rubber, a (meth)acrylic rubber, a butyl rubber, a fluoro rubber, polyethylene oxide, polyvinylpyrrolidone, polyepichlorohydrine, polyphosphazene, poly(meth)acrylonitrile, an ethylene propylene diene copolymer, polyvinylpyridine, chlorosulfonated polyethylene, latex, a polyester resin, a (meth)acrylic resin, a phenol resin, an epoxy resin, polyvinyl alcohol, and/or a (e.g., any suitable) combination thereof.

If (e.g., when) an aqueous binder is used as a binder of the negative electrode, a cellulose-based compound capable of imparting or providing viscosity may be further included. The cellulose-based compound may include at least one selected from among carboxymethyl cellulose, hydroxypropylmethyl cellulose, methyl cellulose, and an alkali metal salt thereof. The alkali metal may include Na, K, and/or Li.

The dry binder may be a polymer material that is capable of being fibrous. For example, the dry binder may be polytetrafluoroethylene, polyvinylidene fluoride, a polyvinylidene fluoride-hexafluoropropylene copolymer, polyethylene oxide, and/or a (e.g., any suitable) combination thereof.

### Negative Electrode Conductive Material

The conductive material (e.g., electrically conductive material or electron conductor) may be used to impart or provide conductivity (e.g., electrical conductivity) to an electrode. Any suitable material that does not cause chemical change (e.g., that does not cause an undesirable chemical change) and is an electron conductive material in a battery may be used. Examples of the conductive material may include: a carbon-based material, such as natural graphite, artificial graphite, carbon black, acetylene black, Ketjen black, a carbon fiber, a carbon nanofiber, and carbon nanotube; a metal-based material including copper, nickel, aluminum, silver, and/or the like, in a form of a metal powder or a metal fiber; a conductive polymer (e.g., an electrically conductive polymer), such as a polyphenylene derivative; and/or a (e.g., any suitable) mixture thereof.

### Negative Electrode Current Collector

The current collector COL2 may use a copper foil, a nickel foil, a stainless steel foil, a titanium foil, a nickel foam, a copper foam, a polymer substrate coated with a conductive metal, and/or a (e.g., any suitable) combination thereof.

### Negative Electrode Active Material

The negative electrode active material in the negative electrode active material layer AML2 may include a material that reversibly intercalates/deintercalates lithium ions, a lithium metal, a lithium metal alloy, a material capable of doping into and de-doping from lithium, or a transition metal oxide.

The material that reversibly intercalates/deintercalates lithium ions may include a carbon-based negative electrode active material, for example, crystalline carbon, amorphous carbon and/or a (e.g., any suitable) combination thereof. The crystalline carbon may be graphite, such as non-shaped (e.g., irregularly shaped), sheet-shaped (e.g., generally sheet-shaped), flake-shaped (e.g., generally flake-shaped), sphere-shaped (e.g., generally sphere-shaped), or fiber-shaped (e.g., generally fiber-shaped) natural graphite and/or artificial graphite. The amorphous carbon may be soft carbon, hard carbon, a mesophase pitch carbide product, calcined coke, and/or the like.

The lithium metal alloy may include an alloy of lithium with a metal selected from among sodium (Na), potassium (K), rubidium (Rb), cesium (Cs), francium (Fr), beryllium (Be), magnesium (Mg), calcium (Ca), strontium (Sr), silicon (Si), antimony (Sb), lead (Pb), indium (In), zinc (Zn), barium (Ba), radium (Ra), germanium (Ge), aluminum (Al), and tin (Sn).

The material capable of doping into and de-doping from lithium may be a Si-based negative electrode active material or a Sn-based negative electrode active material. The Si-based negative electrode active material may include silicon, a silicon-carbon composite, SiOx (where 0<x≤2; e.g., SiO₂), an Si-Q alloy (where Q may be selected from among an alkali metal, an alkaline earth metal, a Group 13 element, a Group 14 element (except for Si), a Group 15 element, a Group 16 element, a transition metal, a rare earth element, and/or a (e.g., any suitable) combination thereof), and/or a (e.g., any suitable) combination thereof. The Sn-based negative electrode active material may be Sn, SnOₖ (where 0<k≤2; e.g., SnO₂), a Sn-based alloy, and/or a (e.g., any suitable) combination thereof.

The silicon-carbon composite may be a composite of silicon and amorphous carbon (e.g., in a form of particles). According to one or more embodiments, the silicon-carbon composite may be in a form of silicon particle or in a form of silicon particle coated with amorphous carbon on the surface thereof. For example, the silicon-carbon composite may include a secondary particle (core), in which primary silicon particles are assembled, and an amorphous carbon coating layer (shell) on (e.g., positioned on) the surface of the secondary particle. The amorphous carbon may also be between the primary silicon particles, and, for example, the primary silicon particles may be coated with the amorphous carbon. The secondary particle may be dispersed in an amorphous carbon matrix.

The silicon-carbon composite may further include crystalline carbon. For example, the silicon-carbon composite may include a core including crystalline carbon and silicon particles and an amorphous carbon coating layer on the surface of the core.

The Si-based negative electrode active material and/or the Sn-based negative electrode active material may be used in combination with a carbon-based negative electrode active material.

### Separator 30

Depending on the type or kind of the rechargeable lithium battery, the separator 30 may be present between the positive electrode 10 and the negative electrode 20. The separator 30 may include polyethylene, polypropylene, polyvinylidene fluoride, or a suitable multilayer of two or more thereof, and may also include a suitable mixed multilayer, such as a polyethylene/polypropylene two-layer separator, a polyethylene/polypropylene/polyethylene three-layer separator, a polypropylene/polyethylene/polypropylene three-layer separator, and/or the like.

The separator 30 may include a porous substrate and a coating layer including an organic material, an inorganic material, and/or a (e.g., any suitable) combination thereof, on a surface (e.g., one surface or two opposite surfaces) of the porous substrate.

The porous substrate may be a polymer film formed of any one polymer selected from among polyolefins, such as polyethylene and polypropylene, polyesters, such as polyethylene terephthalate and polybutylene terephthalate, polyacetal, polyamide, polyimide, polycarbonate, polyether ketone, polyarylether ketone, polyetherimide, polyamide-imide, polybenzimidazole, polyethersulfone, polyphenylene oxide, a cyclic olefin copolymer, polyphenylene sulfide, polyethylene naphthalate, a glass fiber, and polytetrafluoroethylene (e.g., Teflon™), and/or a (e.g., any suitable) copolymer or mixture of two or more thereof.

The organic material may include a polyvinylidene fluoride-based polymer and/or a (meth)acrylic polymer.

The inorganic material may include inorganic particles selected from among Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, boehmite, and/or a (e.g., any suitable) combination thereof, but one or more embodiments of the present disclosure are not limited thereto.

The organic material and the inorganic material may be mixed in one coating layer, or in a stacked form of a coating layer including an organic material and a coating layer including an inorganic material.

### Electrolyte Solution ELL

The electrolyte solution ELL for a rechargeable lithium battery may include a non-aqueous organic solvent and a lithium salt.

The non-aqueous organic solvent may act or serve as a medium for transmitting ions taking part in the electrochemical reaction of a battery.

The non-aqueous organic solvent may be a carbonate-based, ester-based, ether-based, ketone-based, or alcohol-based solvent, an aprotic solvent, and/or a (e.g., any suitable) combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like.

The ester-based solvent may include methyl acetate, ethyl acetate, n-propyl acetate, dimethyl acetate, methyl propionate, ethyl propionate, decanolide, mevalonolactone, valerolactone, caprolactone, and/or the like.

The ether-based solvent may include dibutyl ether, tetraglyme, diglyme, dimethoxyethane, 2-methyltetrahydrofuran, 2,5-dimethyltetrahydrofuran, tetrahydrofuran, and/or the like. In one or more embodiments, the ketone-based solvent may include cyclohexanone, and/or the like. The alcohol-based solvent may include ethyl alcohol, isopropyl alcohol, and/or the like, and the aprotic solvent may include nitriles, such as R—CN (where R may be a C2 to C20 linear, branched, or cyclic hydrocarbon group, and may include a double bond, an aromatic ring, and/or an ether bond); amides, such as dimethylformamide; dioxolanes, such as 1,3-dioxolane and/or 1,4-dioxolane; sulfolanes, and/or the like.

The non-aqueous organic solvents may be used alone or in combination of two or more thereof.

In one or more embodiments, if (e.g., when) using a carbonate-based solvent, a cyclic carbonate and a chain carbonate may be mixed and used, and the cyclic carbonate and the chain carbonate may be mixed in a volume ratio of about 1:1 to about 1:9.

The lithium salt dissolved in the organic solvent supplies lithium ions in a battery, enables a basic operation of a rechargeable lithium battery, and improves the transportation of the lithium ions between the positive electrode and the negative electrode. Examples of the lithium salt may include at least one selected from among LiPF₆, LiBF₄, LiSbF₆, LiAsF₆, LiClO₄, LiAlO₂, LiAlC₄, LiPO₂F₂, LiCl, LiI, LiN(SO₃C₂F₅)₂, Li(FSO₂)₂N (lithium bis(fluorosulfonyl)imide, LiFSI), LiC₄F₉SO₃, LiN(CₓF₂ₓ₊₁SO₂)(C_{y}F_{2y+1}SO₂) (where x and y are integers of 1 to 20), lithium trifluoromethane sulfonate, lithium tetrafluoroethanesulfonate, lithium difluoro(oxalato)borate (LiDFOB), lithium difluorobis(oxalato)phosphate (LiDFBOP), and lithium bis(oxalato) borate (LiBOB).

### Rechargeable Lithium Battery

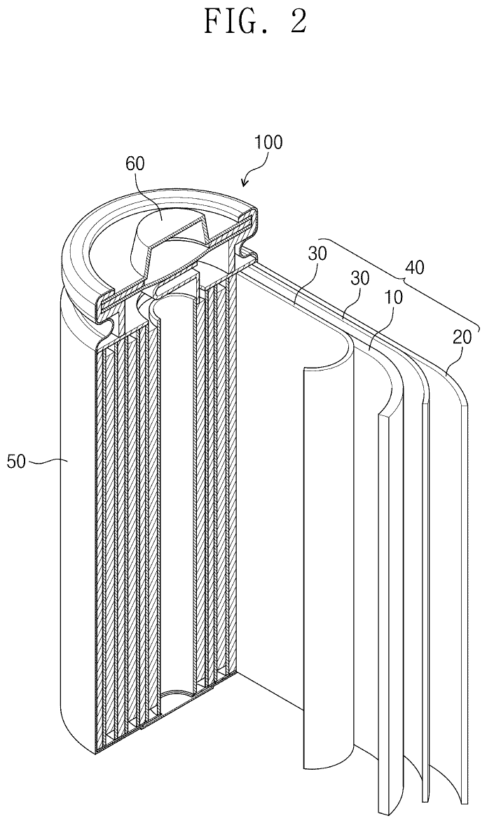

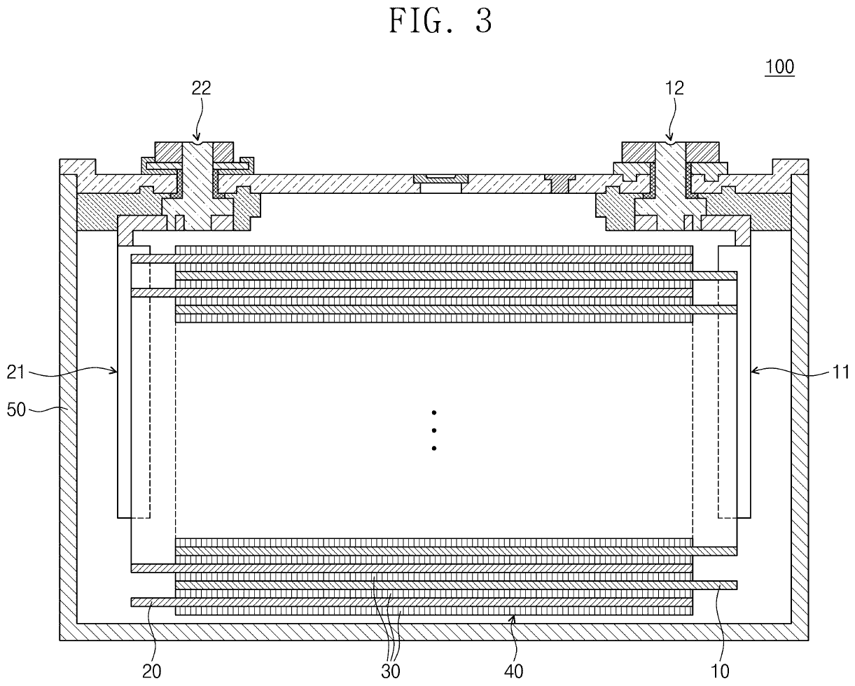

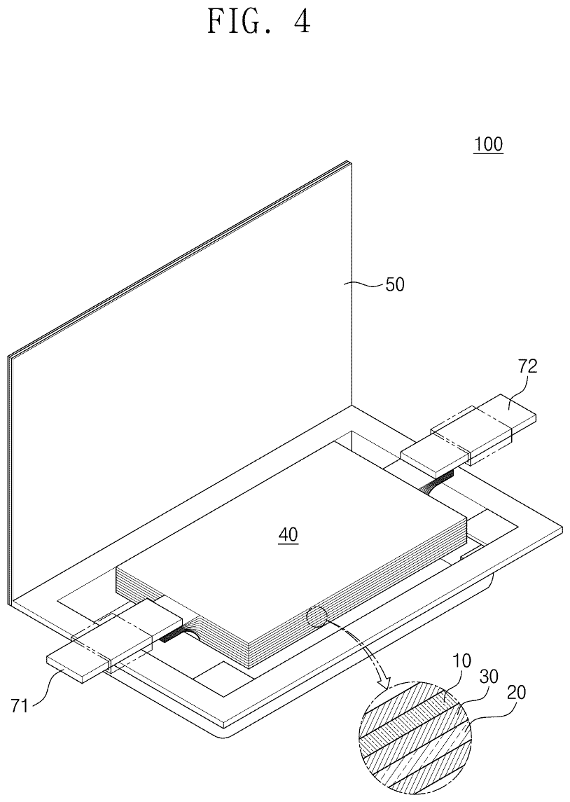

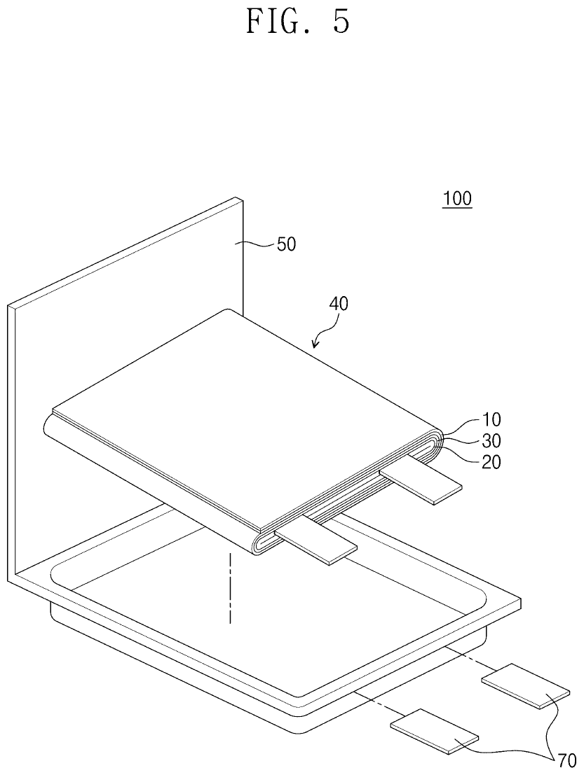

The rechargeable lithium battery may be classified into cylindrical, prismatic, pouch, or coin-type or kind batteries depending on their shape. FIGS. 2-5 each is a schematic view illustrating rechargeable lithium batteries according to one or more embodiments. FIG. 2 shows a cylindrical battery, FIG. 3 shows a prismatic battery, and FIGS. 4 and 5 each shows a pouch-type or kind battery. Referring to FIGS. 2 to 5, the rechargeable lithium battery 100 may include an electrode assembly 40 including a separator 30 between a positive electrode 10 and a negative electrode 20, and a case 50 accommodating the electrode assembly 40. The positive electrode 10, the negative electrode 20, and the separator 30 may be immersed in an electrolyte solution. The rechargeable lithium battery 100 may include a sealing member 60 sealing the case 50, as shown in FIG. 2. In FIG. 3, the rechargeable lithium battery 100 may include a positive electrode lead tab 11, a positive terminal 12, a negative electrode lead tab 21, and a negative terminal 22. As shown in FIGS. 4 and 5, the rechargeable lithium battery 100 may include an electrode tab 70, that is, a positive electrode tab 71 and a negative electrode tab 72, serving as an electrical path for inducing the current formed in the electrode assembly 40 to the outside.

The rechargeable lithium battery according to one or more embodiments may be applied to automobiles, mobile phones, and/or one or more suitable types (or kinds) of electric devices, but embodiments of the present disclosure are not limited thereto.

Hereinafter, a method for fabricating a rechargeable lithium battery according to one or more embodiments of disclosure will be illustrated in more detail.

The rechargeable lithium battery according to one or more embodiments of the present disclosure may include a high nickel-based positive electrode active material. For example, the positive electrode active material according to one or more embodiments may include a compound represented by Chemical Formula 1.

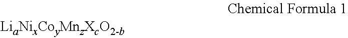

In Chemical Formula 1, 0.8≤a≤1.2, 0.8≤x≤1.0, 0≤y≤0.1, 0≤z≤0.1 0≤c≤0.1, 0≤b≤0.05, x+y+z+c=1, and X may be at least one element selected from among the group consisting of Al, Ti, Mg, Zr, Mo, and Nb.

The capacity of a battery may be improved by including the high nickel-based positive electrode active material.

### Functional Additive

The positive electrode according to one or more embodiments of disclosure may include a functional additive. The functional additive may improve the fabrication processability, stability and/or performance of a rechargeable battery.

In one or more embodiments, surface deterioration due to side reactions in the positive electrode active material may be reduced. For example, complex defects in batteries including slurry gelation, increase in resistance (e.g., electrical resistance), lifetime decrease and increase in gas generation may be solved. That is, complex battery defects such as slurry gelation, increased electrical resistance, reduced lifespan, and/or heightened gas generation may be resolved by the one or embodiments. As a result, a rechargeable battery having improved processability and performance may be provided.

Referring to the layered positive electrode active material as described in one or more embodiments of the present disclosure, a way to improve positive electrode active materials may be to reduce the content (e.g., amount) of expensive cobalt (Co) and/or increase the content (e.g., amount) of nickel (Ni) which exhibits high capacity.

However, as the content (e.g., amount) of nickel (Ni) in the positive electrode active material increases, there may be a defect in that the frequency of side reactions increases due to the reduction of nickel cations on the surface of the active material and the reaction with the atmosphere/moisture. As a result, internal lithium elution and the formation of lithium compounds may cause defects of deteriorating reversible capacity and reducing processability. In one or more embodiments, for a layered active material having the high nickel content (e.g., amount), performance deterioration may begin on the surface of the active material. While a coating process for protecting the surface of the positive electrode active material and a structure stabilization process may be introduced, there may be issues/defects of increasing cost and reducing energy density.

The functional additive according to one or more embodiments of the present disclosure may show improved effects of processability, safety and battery performance through the addition of the functional additive to the positive electrode active material.

The functional additive may contain (or include) a triazole-based compound. The triazole-based compound may refer to a compound including a triazole group. The compound including a triazole group may refer to a triazole derivative. In one or more embodiments, the compound may be a compound including two or more triazole groups.

Triazole may be represented by the molecular formula of C₂H₃N₃, and may be a heterocyclic compound including carbon (C) and nitrogen (N). The triazole compound may have an isomer. For example, 1H-1,2,4-triazole, 1H-1,2,3-triazole, 2H-1,2,3-triazole, and/or 4H-1,2,4-triazole may be included.

By adding a functional additive containing a triazole-based compound to the positive electrode active material, surface deterioration may be reduced, and lifetime characteristics may be improved. For example, a nitrogen atom (N) in the functional additive may combine with the metal ions of the positive electrode active material to reduce surface deterioration. In one or more embodiments, lithium elution may be suppressed or reduced. That is, by incorporating the triazole-based functional additive into the positive electrode active material, surface deterioration may be reduced or minimized, potentially enhancing the battery's lifespan. Specifically, the nitrogen atom in the additive may bond with the metal ions in the electrode material, reducing surface wear. Additionally, lithium elution may be suppressed. Here, even for a thick electrode plate having (with) a (substantially) thick positive active material layer, the fabricating processability, stability, and performance of the battery may be improved. In one or more embodiments, the thickness of the positive active material layer may be about 10 μm to about 30 μm, about 5 μm to about 30 μm, about 10 μm to about 20 μm, about 10 μm to about 40 μm, about 20 μm to about 50 μm, about 40 μm to about 50 μm, about 60 μm to about 65 μm, or about 60 μm to about 70 μm.

The content (e.g., amount) of the functional additive (add) may be about 0.01 parts by weight to about 0.1 parts by weight, about 0.02 parts by weight to about 0.05 parts by weight, or about 0.03 parts by weight to about 0.06 parts by weight on the basis of about 100 parts (e.g., based on 100 parts) by weight of the positive electrode active material. If (e.g., when) the functional additive is excessive, the resistance (e.g., electrical resistance) of an electrode plate may increase, and performance may be deteriorated. By satisfying the above numerical range, the performance of the positive electrode active material, including fabricating processability, stability, and capacity, may be comprehensively improved.

Hereinafter, the triazole-based compound according to one or more embodiments of disclosure will be illustrated in more detail.

In one or more embodiments, the functional additive may include a compound represented by Chemical Formula 2.

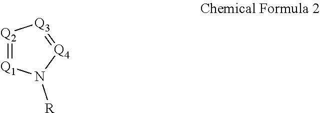

In Chemical Formula 2,

any two selected from among Q₁, Q₂, Q₃, and Q₄ may (each) be nitrogen, and the remainder may (each) be carbon.

R may be selected from among the group consisting of hydrogen, deuterium, a halogen group, a substituted or unsubstituted alkyl group, a substituted or unsubstituted alkenyl group, a substituted or unsubstituted alkynyl group, a substituted or unsubstituted hydrocarbon ring group, a substituted or unsubstituted cyclic alkyl group, a substituted or unsubstituted aryl group, and a substituted or unsubstituted heterocyclic group.

In one or more embodiments, the functional additive may include a compound represented by Chemical Formula 3.

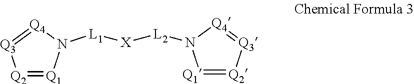

In Chemical Formula 3,

any two selected from among Q₁, Q₂, Q₃, and Q₄ may (each) be nitrogen, and the remainder may (each) be carbon.

Any two selected from among Q₁′, Q₂′, Q₃′, and Q₄′ may (each) be nitrogen, and the remainder may (each) be carbon.

L₁, and L₂ may each independently be any one selected from among a direct linkage, a substituted or unsubstituted alkylene group of 1 to 6 carbon atoms, an alkenylene group, and an alkynylene group, and X may be any one selected from among sulfonyl group, a sulfinyl group, a sulfone group, and a carbonyl group.

In one or more embodiments, the functional additive may include a compound represented by Chemical Formula 4.

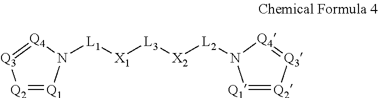

In Chemical Formula 4,

any two selected from among Q₁, Q₂, Q₃, and Q₄ may (each) be nitrogen, and the remainder may (each) be carbon.

Any two selected from among Q₁′, Q₂′, Q₃′, and Q₄′ may (each) be nitrogen, and the remainder may (each) be carbon.

L₁, L₂, and L₃ may each independently be any one selected from among a direct linkage, a substituted or unsubstituted alkylene group of 1 to 6 carbon atoms, an alkenylene group, and an alkynylene group, and X₁ and X₂ may each independently be any one selected from among a sulfonyl group, a sulfinyl group, a sulfone group, and a carbonyl group.

In one or more embodiments, the functional additive may include a compound represented by Chemical Formula 5.

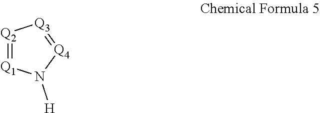

In Chemical Formula 5,

any two selected from among Q₁, Q₂, Q₃, and Q₄ may (each) be nitrogen, and the remainder may (each) be carbon.

In one or more embodiments, the functional additive may include a compound represented by Chemical Formula 6.

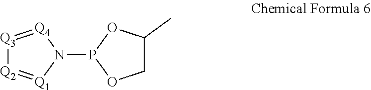

In Chemical Formula 6,

any two selected from among Q₁, Q₂, Q₃, and Q₄ may (each) be nitrogen, and the remainder may (each) be carbon.

In one or more embodiments, the functional additive may include a compound represented by Chemical Formula 7.

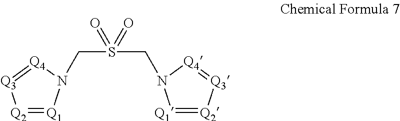

In Chemical Formula 7,

any two selected from among Q₁, Q₂, Q₃, and Q₄ may (each) be nitrogen, and the remainder may (each) be carbon.

Any two selected from among Q₁′, Q₂′, Q₃′, and Q₄′ may (each) be nitrogen, and the remainder may (each) be carbon.

In one or more embodiments, the functional additive may include a compound represented by Chemical Formula 8.

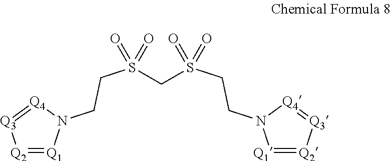

In Chemical Formula 8,

any two selected from among Q₁, Q₂, Q₃, and Q₄ may (each) be nitrogen, and the remainder may (each) be carbon.

Any two selected from among Q₁′, Q₂′, Q₃′, and Q₄′ may (each) be nitrogen, and the remainder may (each) be carbon.

The examples above are illustrations, and one or more embodiments of the present disclosure are not limited thereto.

### Electrolyte Layer

In one or more embodiments, a separate electrolyte layer may be included (or arranged) between the positive electrode and the negative electrode. The electrolyte layer may include the separator 30 and the electrolyte solution ELL. For example, the entire structure including the separator and the electrolyte solution may be collectively referred to as the electrolyte layer. The electrolyte layer may be formed or provided in the form of a film having a certain thickness.

In one or more embodiments, the electrolyte layer may be a gel polymer electrolyte layer including a gel polymer electrolyte. If (e.g., when) the gel polymer electrolyte (or semi-solid electrolyte) is included, a gel polymer electrolyte containing a liquid electrolyte and a crosslinked polymer in the pores of a porous base constituting the separator 30 may be included. In the structure, a crosslinked polymer network having a crosslinked structure may be impregnated with the liquid electrolyte to prevent or reduce the liquid electrolyte from leaking out.

In one or more embodiments, the electrolyte layer may be a solid electrolyte layer including a solid electrolyte. The solid electrolyte layer may replace the role of the separator 30 and the electrolyte solution ELL.

For a rechargeable battery containing a gel polymer electrolyte (or semi-solid electrolyte) or a solid electrolyte, the electrolyte layer may replace the role of the separator 30 and the electrolyte solution ELL.

### Method for Fabricating Rechargeable Lithium Battery

Hereinafter, a method for fabricating a rechargeable lithium battery according to one or more embodiments will be illustrated.

A method for fabricating a rechargeable lithium battery may include a step (e.g., act or task) of manufacturing a positive electrode, a step (e.g., act or task) of manufacturing a negative electrode, and a step (e.g., act or task) of combining the positive electrode and the negative electrode to fabricate a battery.

For example, the step (e.g., act or task) of manufacturing the positive electrode may include a step (e.g., act or task) of mixing a positive electrode active material, a conductive material, a binder, and a functional additive to prepare a positive electrode active material slurry, and a step (e.g., act or task) of applying the positive electrode active material slurry on a current collector to form or provide a positive electrode active material layer. The functional additive may include a compound containing a triazole group.

### Step (e.g., Act of Task) of Manufacturing Positive Electrode

A positive electrode active material slurry may be prepared by mixing a positive electrode active material, a conductive material, a binder, and a functional additive. The mixing method may be any suitable methods that may be used by a person skilled in the art, including a wet or dry method, and is not limited to a specific method.

In the step (e.g., act or task) of preparing the positive electrode active material slurry, the above-described materials may be used as the positive electrode active material, conductive material, and binder. For example, a high nickel-based positive electrode active material may be used as the positive electrode active material.

In the step (e.g., act or task) of preparing the positive electrode active material slurry, the functional additive may be added. The functional additive may be the same (or substantially the same) as described in one or more embodiments of the present disclosure, and the explanation thereof will not be provided, and the step (e.g., act or task) of manufacturing the positive electrode will be illustrated in more detail.

For example, by adding the functional additive in the step (e.g., act or task) of mixing and preparing the positive electrode active material slurry, performance deterioration defect generated during manufacturing the positive electrode may be solved. As a result, a positive electrode having improved processability and performance may be provided. The content (e.g., amount) of the functional additive added may be about 0.01 parts by weight to about 0.1 parts by weight, about 0.02 parts by weight to about 0.05 parts by weight, or about 0.03 parts by weight to about 0.06 parts by weight on the basis of about 100 parts (e.g., based on 100 parts) by weight of the positive electrode active material.

For example, in the step (e.g., act or task) of mixing the positive electrode active material slurry, defects including the increase in resistance (e.g., electrical resistance) and gas generation due to side reactions with the atmosphere/moisture may be solved. Defects, such as the generation of lithium compounds, binder denaturation, and the agglomeration of a conductive material, resulting in the gelation of the positive electrode active material slurry, may be effectively solved.

In one or more embodiments, because the functional additive is mixed uniformly (e.g., substantially uniformly) in the positive electrode active material slurry, a positive electrode active material layer in which the functional additive is dispersed uniformly (e.g., substantially uniformly) may be formed. In one or more embodiments, for a thick electrode plate having a thick positive active material layer, the fabrication processability and stability may be improved. As a result, a battery having improved fabrication processability, stability and, capacity may be obtained. In one or more embodiments, the thickness of the positive electrode active material layer may be about 10 μm to about 30 μm, about 5 μm to about 30 μm, about 10 μm to about 20 μm, about 10 μm to about 40 μm, about 20 μm to about 50 μm, about 40 μm to about 50 μm, about 60 μm to about 65 μm, or about 60 μm to about 70 μm.

Afterwards, the positive electrode active material slurry may be applied on a positive electrode current collector, dried, and rolled to form or provide a positive electrode active material layer.

### Step (e.g., Act or Task) of Manufacturing Negative Electrode

A negative electrode active material slurry may be prepared by mixing a negative electrode active material, a binder, and a conductive material. The negative electrode active material slurry prepared may be applied on a negative electrode current collector, dried, and rolled to form or provide a negative electrode.

### Step (e.g., Act or Task) of Fabricating Battery

A completed rechargeable lithium battery may be fabricated by combining the positive electrode and the negative electrode manufactured as described in one or more embodiments of the present disclosure. A separator may be positioned (or arranged) between the positive electrode and the negative electrode, and an electrolyte solution may be injected to fabricate a battery. In one or more embodiments, an electrolyte layer may be provided (or arranged) between the positive electrode and the negative electrode to fabricate a battery having a stack type or kind.

Any suitable batteries that have a positive electrode active material layer, such as a general lithium ion battery, an all-solid battery, and a semi-solid battery, may be applied. In one or more embodiments, the shape of the completed battery is not limited and may be fabricated in one or more suitable shapes, such as prismatic, pouch-type or kind, and/or cylindrical shapes (e.g., generally cylindrical shapes).

Hereinafter, embodiments/examples for performing present disclosure will be described. The present disclosure will include not only be the above-described embodiments, but also embodiments that may be simply changed in design or readily changed. In one or more embodiments, present disclosure will include technologies that may be easily modified and implemented using one or more embodiments/examples. Accordingly, the scope of the present disclosure should not be limited to one or more embodiments/examples, but should be determined by the appended claims described in more detail later as well as the equivalents thereof.

### Example 1: Preparation of Positive Electrode Slurry

98.45 wt % of a LiNi_{0.94}Co_{0.04}Al_{0.02}O₂ positive electrode active material, 1.2 wt % of a polyvinylidene fluoride binder, and 0.35 wt % of a carbon nanotube conductive material were mixed in a N-methylpyrrolidone solvent to prepare a positive electrode active material slurry. To the positive electrode active material slurry, a functional additive containing a compound of Chemical Formula A-1 was added.

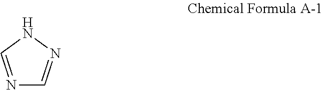

### Example 2: Preparation of Positive Electrode Slurry

To the positive electrode active material slurry, a functional additive containing a compound of Chemical Formula A-2 was added.

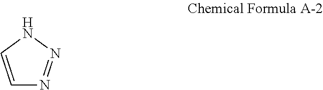

A positive electrode slurry was prepared by the same (e.g., substantially the same) method as in Example 1 except for this functional additive containing a compound of Chemical Formula A-2.

### Example 3: Preparation of Positive Electrode Slurry

To the positive electrode active material slurry, a functional additive containing a compound of Chemical Formula A-3 was added.

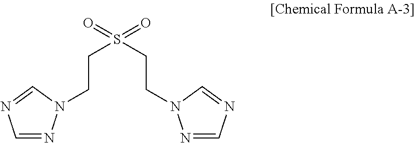 [SMILES: O=S(=O)(CCn1cncn1)CCn1cncn1]

A positive electrode slurry was prepared by the same (e.g., substantially the same) method as in Example 1 except for this functional additive containing a compound of Chemical Formula A-3.

### Example 4: Preparation of Positive Electrode Slurry

To the positive electrode active material slurry, a functional additive containing a compound of Chemical Formula A-4 was added.

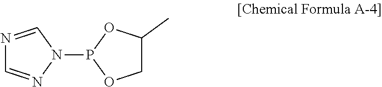

A positive electrode slurry was prepared by the same (e.g., substantially the same) method as in Example 1 except for this functional additive containing a compound of Chemical Formula A-4.

### Example 5: Preparation of Positive Electrode Slurry

To the positive electrode active material slurry, a functional additive containing a compound of Chemical Formula A-5 was added.

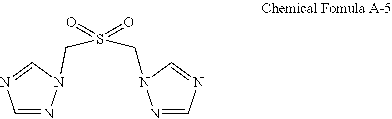 [SMILES: O=S(=O)(Cn1cncn1)Cn1cncn1]

A positive electrode slurry was prepared by the same (e.g., substantially the same) method as in Example 1 except for this functional additive containing a compound of Chemical Formula A-5.

### Example 6: Preparation of Positive Electrode Slurry

To the positive electrode active material slurry, a functional additive containing a compound of Chemical Formula A-6 was added.

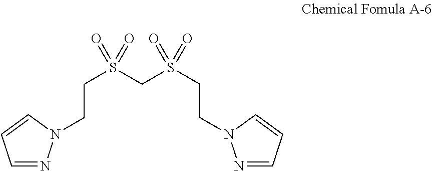 [SMILES: O=S(=O)(CCn1cccn1)CS(=O)(=O)CCn1cccn1]

A positive electrode slurry was prepared by the same (e.g., substantially the same) method as in Example 1 except for this functional additive containing a compound of Chemical Formula A-6.

### Example 7: Preparation of Positive Electrode Slurry

A positive electrode slurry was prepared using the same (e.g., substantially the same) functional additive as in Example 1 except for increasing the content (e.g., amount) of the functional additive.

### Comparative Example 1: Preparation of Positive Electrode Slurry

A functional additive containing oxalic acid was added to the positive electrode active material slurry. A positive electrode slurry was prepared by the same (e.g., substantially the same) method as in Example 1 except for this functional additive containing oxalic acid.

### Comparative Example 2: Preparation of Positive Electrode Slurry

A functional additive containing oxalic acid was added to the positive electrode active material slurry. A positive electrode slurry was prepared by the same (e.g., substantially the same) method as in Comparative Example 1 except for changing the content (e.g., amount) of the functional additive.

The Examples and Comparative Examples in the present disclosure prepared are shown in Table 1. The contents (e.g., amounts) of the functional additive are shown by the parts by weight of the functional additive relative to about 100 parts (e.g., based on 100 parts) by weight of the positive electrode active material.

TABLE 1

| | Functional additive (parts by weight relative to |
|---|---|
| | positive electrode active material) |
| Example 1 | Triazole 0.035 parts by weight |
| Example 2 | Triazole 0.035 parts by weight |
| Example 3 | Triazole 0.035 parts by weight |
| Example 4 | Triazole 0.035 parts by weight |
| Example 5 | Triazole 0.035 parts by weight |
| Example 6 | Triazole 0.035 parts by weight |
| Example 7 | Triazole 0.2 parts by weight |
| Comparative Example 1 | Oxalic acid 0.1 parts by weight |
| Comparative Example 2 | Oxalic acid 0.15 parts by weight |

### Evaluation Example 1: Viscosity Change During Standing Slurry Stirring

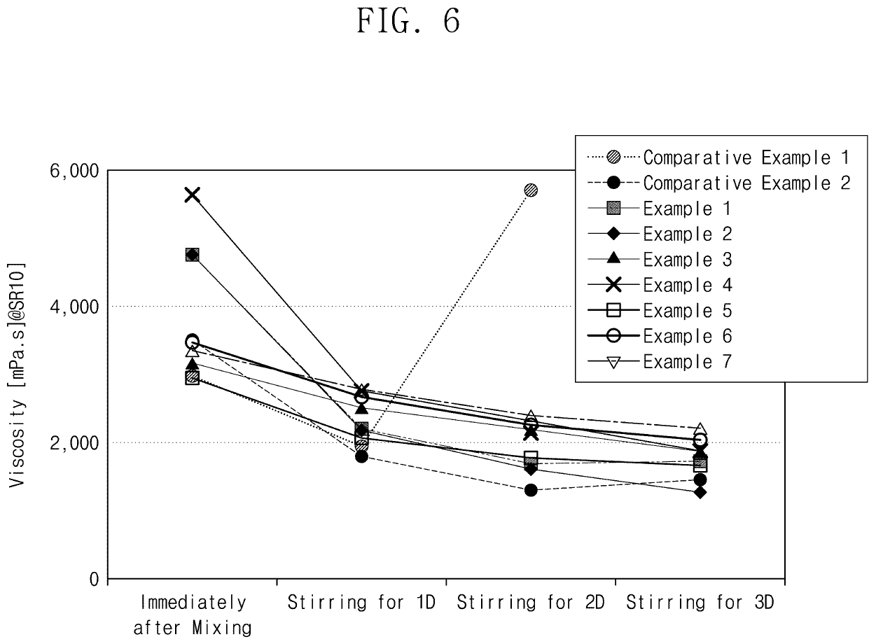

After preparing slurries by adding a functional additive, the slurries were stood in a low-stirring state, and the viscosity change of the slurries was observed. That is, after the slurries each with its corresponding functional additive were prepared, the slurries were then left in the low-stirring state to observe any changes in viscosity. The results are shown in Table 2 and FIG. 6.

TABLE 2

| | Viscosity (mPa · s) | | | |
|---|---|---|---|---|
| | Immediately | Stirring | Stirring | Stirring |
| | after mixing | for 1 day | for 2 days | for 3 days |
| Example 1 | 4,758 | 2,216 | 1,699 | 1,718 |
| Example 2 | 4,763 | 2,175 | 1,605 | 1,267 |
| Example 3 | 3,173 | 2,516 | 2,191 | 1,906 |
| Example 4 | 5,632 | 2,758 | 2,135 | 1,870 |
| Example 5 | 2,946 | 2,069 | 1,777 | 1,675 |
| Example 6 | 3,469 | 2,683 | 2,261 | 2,048 |
| Example 7 | 3,352 | 2,783 | 2,399 | 2,217 |
| Comparative | 2,988 | 1,952 | 5,720 | — |
| Example 1 | | | | |
| Comparative | 3,513 | 1,805 | 1,312 | 1,465 |
| Example 2 | | | | |

It may be found that the positive electrode active material slurries according to the Examples of the present disclosure each have reduced viscosity according to the lapse of stirring time after mixing.

In contrast, as for Comparative Example 1, it may be found that agglomeration occurs due to the denaturation of the binder during standing stirring, and the viscosity rapidly increases. It may be found that a larger amount of the additive is necessary as in Comparative Example 2 in order to maintain appropriate or suitable viscosity.

### Evaluation Example 2: Analysis on the Surface of Active Material

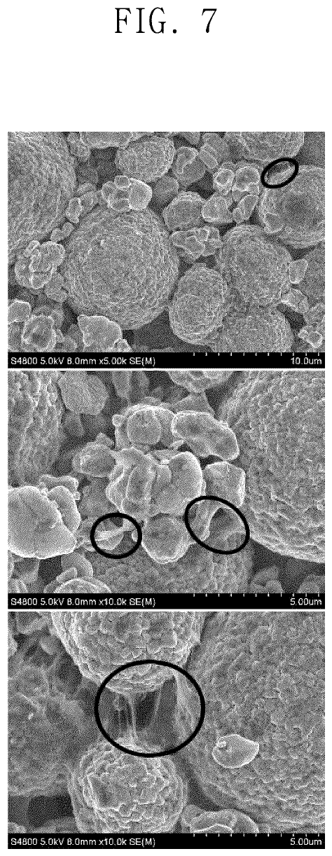

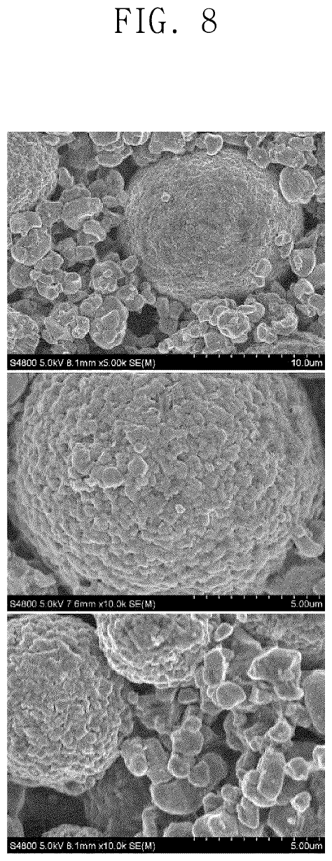

FIG. 7 is the scanning electron microscope (SEM) images analyzing the surface of the active material of Comparative Example 2. FIG. 8 is the scanning electron microscope (SEM) images analyzing the surface of the active material of Example 3.

Referring to FIG. 7, it may be confirmed that the agglomeration of a conductive material occurs, and aggregates (e.g., agglomerates) are formed in the positive electrode according to the Comparative Example. Referring to FIG. 8, the formation of aggregates (e.g., agglomerates) is little at the surface of the active material according to the Example of disclosure, and the surface of the active material is substantially uniform.

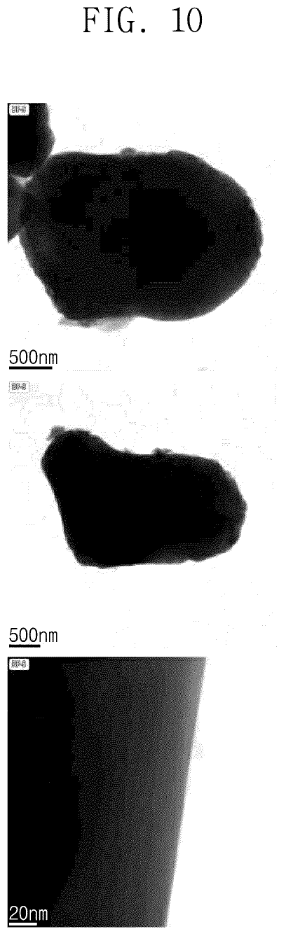

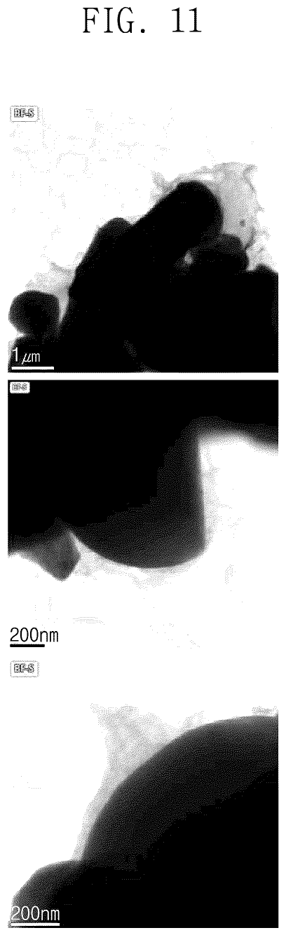

FIG. 10 is the transmissive electron microscope (TEM) images analyzing the surface of the active material of Comparative Example 2. FIG. 11 is the transmissive electron microscope (SEM) images analyzing the surface of the active material of Example 3.

When FIG. 10 and FIG. 11 are compared, it may be confirmed that a layer having several to tens nanometers in size is present only at the surface of the active material according to the Example of the present disclosure.

### Evaluation Example 3: Evaluation of Resistance

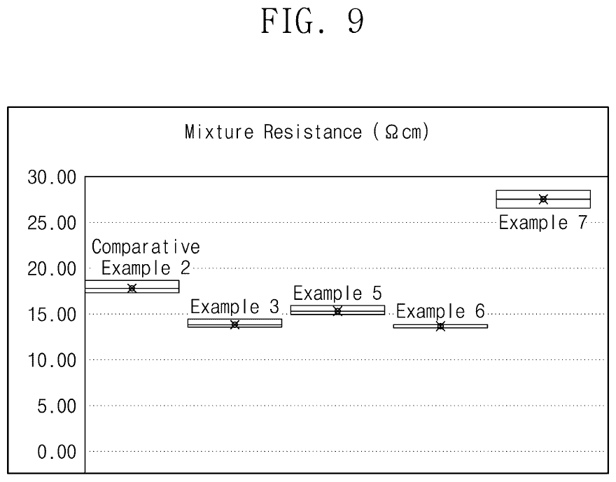

The resistance (e.g., electrical resistance) of a positive electrode mixture was measured for various (some) Examples and Comparative Examples and shown in FIG. 9. It may be found that the resistance of a positive electrode mixture according to each of the Examples of the present disclosure is low compared to the Comparative Example. However, it may be found that if an excessive amount of the functional additive is included as in Example 7, the resistance increases, and the performance is rather deteriorated.

### Evaluation Example 4: Evaluation of Lifetime

Coin cells were fabricated using the positive electrodes manufactured by the positive electrode active material slurries after stirring for 1 D of the Examples and Comparative Examples, and lifetime characteristics were evaluated. The evaluation results on lifetime capacity retention are shown in Table 3. The evaluation results on storage properties are shown in Table 4.

TABLE 3

| | 55° C. lifetime capacity |
|---|---|
| | retention (%, @100 cycle) |
| Example 1 | 92.9 |
| Example 2 | 93.5 |
| Example 3 | 93.8 |
| Example 4 | 93.3 |
| Example 5 | 93.6 |
| Example 6 | 93.4 |
| Example 7 | 93.6 |
| Comparative Example 1 | 86.0 |
| Comparative Example 2 | 86.7 |

TABLE 4

| | 90° C. storage properties |
|---|---|
| | (Full cell CID open time) |
| Example 3 | 51 |
| Example 5 | 44 |
| Example 6 | 46 |
| Example 7 | 48 |
| Comparative Example 1 | 20 |
| Comparative Example 2 | 25 |

Referring to Tables 3 and 4, it may be found that the batteries according to the Examples have excellent or suitable lifetime characteristics and safety in contrast to those of the Comparative Examples.

### Evaluation Example 5: XPS Analysis Results

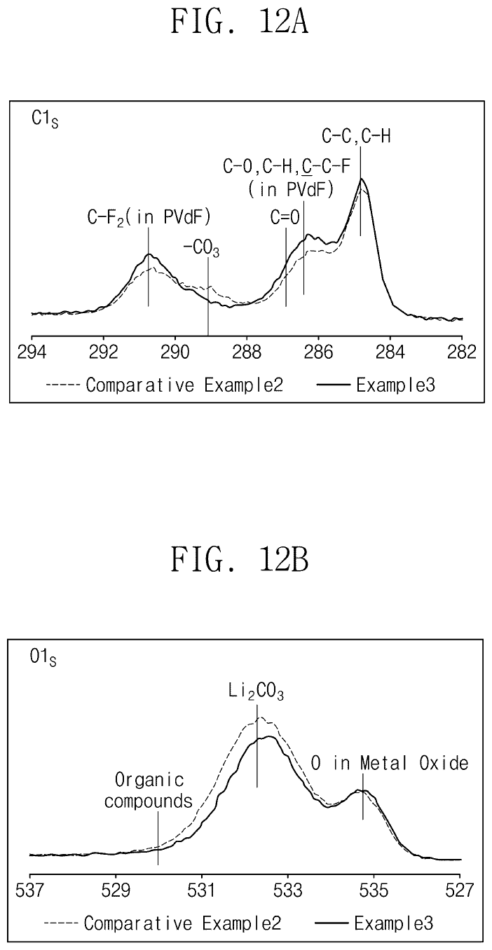

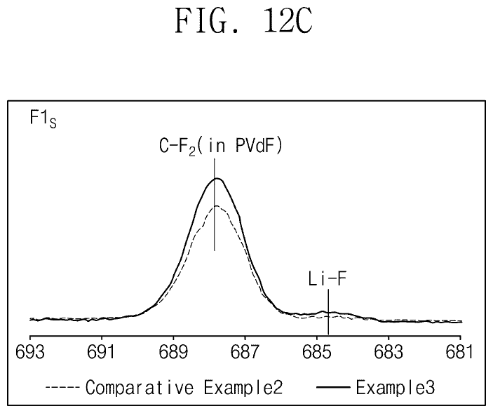

The surfaces of the positive electrode active materials according to the Example and Comparative Example were analyzed by XPS. The results are shown in FIGS. 12A, 12B, and 12C.

Referring to FIGS. 12A and 12B, it may be confirmed that the intensity of the peak related to (—CO₃) of each of the Example is lower compared to the Comparative Example. This should indicate that a surface protective layer was formed on the active material by the functional additive. For example, it should indicate that lithium elution from the active material was suppressed or reduced during mixing a slurry, and the production of lithium carbonate (Li₂CO₃) was suppressed or reduced. That is, FIGS. 12A and 12B show that the intensity of the peak related to (—CO₃) is lower in the Examples compared to the Comparative Examples. This suggests that the surface protective layer was formed on the active material by the functional additive, which suppressed lithium elution and reduced the production of lithium carbonate (Li2CO₃) during slurry mixing.

Referring to FIGS. 12A and 12C, the C-F peak increased in the case of the Example compared to the Comparative Example, which suggests that denaturation due to the de-hydrofluorination reaction (de-HF) of a PVDF binder was reduced, and as a result, the formation of aggregates (e.g., agglomerates) was also suppressed or reduced.

As a result, it may be found that the stability was improved due to the surface protection effect of the positive electrode active material by including the functional additive.

A positive electrode according to one or more embodiments has improved surface protection effect of a positive electrode active material, and the lifetime characteristics and output properties of a rechargeable battery may be improved.

A fabrication method according to one or more embodiments may solve or address the defects of deteriorating the processability, stability and battery performance of a rechargeable battery.

A battery manufacturing device, a battery management system (BMS) device, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

A person of ordinary skill in the art, in view of the present disclosure in its entirety, would appreciate that each suitable feature of the various embodiments of the present disclosure may be combined or combined with each other, partially or entirely, and may be technically interlocked and operated in various suitable ways, and each embodiment may be implemented independently of each other or in conjunction with each other in any suitable manner unless otherwise stated or implied.

Although one or more embodiments of disclosure have been described, it is understood that the present disclosure should not be limited to these embodiments but one or more suitable changes and modifications can be made by one ordinary skilled in the art within the spirit and scope of present disclosure as hereinafter claimed and equivalents thereof.

What is claimed is:

1. A positive electrode active material slurry, comprising:
a functional additive;
a positive electrode active material;
a conductive material; and
a binder,
wherein the functional additive comprises a compound containing a triazole group.

2. The positive electrode active material slurry as claimed in claim 1, wherein the positive electrode active material comprises a compound of Chemical Formula 1: 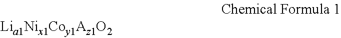
in Chemical Formula 1, 0.8≤a≤1.2, 0.8≤x≤1.0, 0≤y≤0.1, 0≤z≤0.1 0≤c≤0.1, 0≤b≤0.05, x+y+z+c=1, and X is at least one element selected from among the group consisting of Al, Ti, Mg, Zr, Mo, and Nb.

3. The positive electrode active material slurry as claimed in claim 1, wherein the compound comprises two or more triazole groups.

4. The positive electrode active material slurry as claimed in claim 1, wherein the functional additive is about 0.01 parts by weight to about 0.1 parts by weight in content based on 100 parts by weight of the positive electrode active material.

5. The positive electrode active material slurry as claimed in claim 1, wherein the compound is represented by Chemical Formula 2: 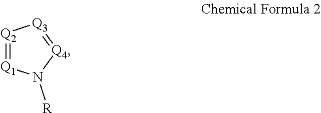
in Chemical Formula 2,
any two selected from among Q₁, Q₂, Q₃, and Q₄ are each nitrogen, and the remainder is each carbon, and
R is selected from among the group consisting of hydrogen, deuterium, a halogen group, a substituted or unsubstituted alkyl group, a substituted or unsubstituted alkenyl group, a substituted or unsubstituted alkynyl group, a substituted or unsubstituted hydrocarbon ring group, a substituted or unsubstituted cyclic alkyl group, a substituted or unsubstituted aryl group, and a substituted or unsubstituted heterocyclic group.

6. The positive electrode active material slurry as claimed in claim 1, wherein the compound is represented by Chemical Formula 3: 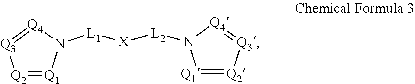
in Chemical Formula 3,
any two selected from among Q₁, Q₂, Q₃, and Q₄ are each nitrogen, and the remainder is each carbon,
any two selected from among Q₁′, Q₂′, Q₃′, and Q₄′ are each nitrogen, and the remainder is each carbon,
L₁, and L₂ are each independently any one selected from among a direct linkage, a substituted or unsubstituted alkylene group of 1 to 6 carbon atoms, an alkenylene group, and an alkynylene group, and
X is any one selected from among a sulfonyl group, a sulfinyl group, a sulfone group, and a carbonyl group.

7. The positive electrode active material slurry as claimed in claim 1, wherein the compound is represented by Chemical Formula 4: 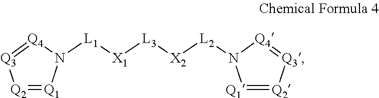
in Chemical Formula 4,
any two selected from among Q₁, Q₂, Q₃, and Q₄ are each nitrogen, and the remainder is each carbon,
any two selected from among Q₁′, Q₂′, Q₃′, and Q₄′ are each nitrogen, and the remainder is each carbon,
L₁, L₂, and L₃ are each independently any one selected from among a direct linkage, a substituted or unsubstituted alkylene group of 1 to 6 carbon atoms, an alkenylene group, and an alkynylene group, and
X₁ and X₂ are each independently any one selected from among a sulfonyl group, a sulfinyl group, a sulfone group, and a carbonyl group.

8. The positive electrode active material slurry as claimed in claim 1, wherein the compound is represented by Chemical Formula 5: 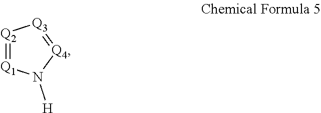
in Chemical Formula 5,
any two selected from among Q₁, Q₂, Q₃, and Q₄ are each nitrogen, and the remainder is each carbon.

9. The positive electrode active material slurry as claimed in claim 1, wherein the compound is represented by Chemical Formula 6: 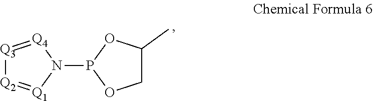
in Chemical Formula 6,
any two selected from among Q₁, Q₂, Q₃, and Q₄ are each nitrogen, and the remainder is each carbon.

10. The positive electrode active material slurry as claimed in claim 1, wherein the compound is represented by Chemical Formula 7: 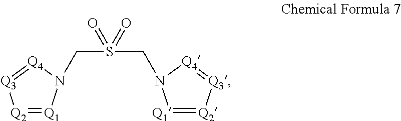
in Chemical Formula 7,
any two selected from among Q₁, Q₂, Q₃, and Q₄ are each nitrogen, and the remainder is each carbon, and
any two selected from among Q₁′, Q₂′, Q₃′, and Q₄′ are each nitrogen, and the remainder is each carbon.

11. The positive electrode active material slurry as claimed in claim 1, wherein the compound is represented by Chemical Formula 8: 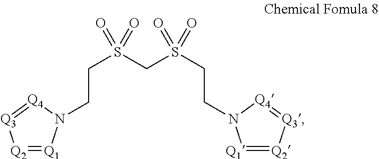
in Chemical Formula 8,
any two selected from among Q₁, Q₂, Q₃, and Q₄ are each nitrogen, and the remainder is each carbon, and
any two selected from among Q₁′, Q₂′, Q₃′, and Q₄′ are each nitrogen, and the remainder is each carbon.

12. A rechargeable lithium battery comprising:
a positive electrode;
a negative electrode; and
an electrolyte layer between the positive electrode and the negative electrode,
wherein,
the positive electrode comprises a positive electrode current collector and a positive electrode active material layer on the positive electrode current collector,
the negative electrode comprises a negative electrode current collector and a negative electrode active material layer on the negative electrode current collector, and
the positive electrode active material layer comprises the positive electrode active material slurry as claimed in claim 1.

13. The rechargeable lithium battery as claimed in claim 12, wherein the positive electrode active material layer is about 60 μm to about 70 μm in thickness.

14. The rechargeable lithium battery as claimed in claim 12, wherein the functional additive in the positive electrode active material layer is about 0.01 parts by weight to about 0.1 parts by weight in content based on 100 parts by weight of the positive electrode active material in the positive electrode active material layer.

15. The rechargeable lithium battery as claimed in claim 12,
wherein the positive electrode active material layer comprises a conductive material, and
the conductive material comprises: a carbon-based material comprising natural graphite, artificial graphite, carbon black, acetylene black, Ketjen black, carbon fiber, carbon nano-fiber, and carbon nano-tube; a metal-based material comprising copper, nickel, aluminum, or silver and having a metal powder or metal fiber form; a conductive polymer comprising a polyphenylene derivative; or a mixture thereof.

16. The rechargeable lithium battery as claimed in claim 12, wherein the electrolyte layer comprises a gel polymer electrolyte.

17. The rechargeable lithium battery as claimed in claim 12, wherein the electrolyte layer comprises a solid electrolyte.

18. A method comprising:
manufacturing a positive electrode;
manufacturing a negative electrode; and
combining the positive electrode and the negative electrode to fabricate a battery,
wherein the manufacturing of the positive electrode comprises:
mixing a positive electrode active material, a conductive material, a binder, and a functional additive to prepare a positive electrode active material slurry; and
applying the positive electrode active material slurry on a current collector to form a positive electrode active material layer, and
the functional additive comprises a compound containing a triazole group, and
wherein the method is a method for fabricating a rechargeable lithium battery.

19. The method as claimed in claim 18, wherein the functional additive is about 0.01 parts by weight to about 0.1 parts by weight in content based on 100 parts by weight of the positive electrode active material.

20. The method for fabricating a rechargeable lithium battery as claimed in claim 18, wherein the positive electrode active material comprises a compound of Chemical Formula 1: 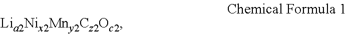
in Chemical Formula 1, 0.8≤a≤1.2, 0.8≤x≤1.0, 0≤y≤0.1, 0≤z≤0.1 0≤c≤0.1, 0≤b≤0.05, x+y+z+c=1, and X is at least one element selected from among the group consisting of Al, Ti, Mg, Zr, Mo, and Nb.
